(19) United States
(12) Patent Application Publication
(10) Pub. No.: US 2019/0128875 A1
(43) Pub. Date: May 2, 2019

(54) EXCREMENT DETECTING DISC AND A MANUFACTURING METHOD THEREOF

(71) Applicant: Xiamen Brana Design Co., Ltd., Xiamen (CN)

(72) Inventor: Zhongtang Wang, Xiamen (CN)

(21) Appl. No.: 16/169,974

(22) Filed: Oct. 24, 2018

(30) Foreign Application Priority Data
Oct. 26, 2017 (CN) 201711087603.X

(51) Int. Cl. G01N 33/52; G01N 33/493

(52) U.S. Cl. CPC G01N 33/525; G01N 33/72; G01N 33/493

(57) ABSTRACT

An excrement detecting disc and a manufacturing method thereof are disclosed. An excrement detecting reagent block and a control reagent block are pasted on fan-shaped blades with spoke-like arrangement on the detecting disc to accommodate more detection items. The spaces are located between the blades to avoid the detection items interfering with each other. The widths and shapes of the blades are changed, and the personalized detection items and the general detection items coexist in the detecting disc. Various types of detection items are collected. A negative control is set for each detection item to improve the reliability of the detection results.

### BACKGROUND OF THE INVENTION

### 1. Field of the Invention

This disclosure relates to a medical consumable for in vitro diagnosis in particular to an excrement detecting disc for qualitatively or quantitatively detecting a specific component of excrement such as urine and feces, and a manufacturing method of an excrement detecting disc.

### 2. Description of the Prior Art

Human excretions, especially urine and feces, contain a large amount of physiological and pathological information. Therefore, urine and stool examinations have become clinical routine check item.

With the progress of the dry chemical test strips and cooperating scanning test equipment related to the routine detection items of urine, the routine examinations of urine are basically automated. The conventional automated detection of feces has also made significant progress in recent years.

The detection reagent “all-in-one” of the detection item of the urine or feces is collected on the test strip to improve the detection efficiency. But there are still deficiencies including:

(1) The number of detection items increases, the length of test strips increases, and the corresponding detection equipment increases in volume so that an increase of the amount of excrement samples is required;

(2) Interference between reagents for the detection items, affecting the accuracy of the results;

(3) Items with significantly different detection principles cannot coexist in the same test strip, for example, the needs to heating, cooling, and add specific reagents, etc.;

(4) The test strip moves back and forth, left and right, and moves more than the circumference, which consumes more energy and has lower efficiency;

(5) Multiple tests share a blank control, and the error is large, making it difficult to achieve quantitative detection;

In view of the above, the test strip is designed into a disc having a spoke-like shape, which completely separates the interference between the reagents, multiplies the space for installation of the detection items, and the personalized detection items and the general detection items can be mashed up and coexist to assist the automation of the manual operation. The results are more accurate and the work is more efficient.

### SUMMARY OF THE INVENTION

The object of this disclosure is to provide an excrement detecting disc for detecting specific components of excrement such as urine and feces, and a manufacturing method thereof For avoiding mutual interference between reagents of different detection items, the personalized detection items and the general detection items are mashed and coexist in the same detecting disc to improve work efficiency.

To achieve the above object, the solution according to this disclosure is:

An excrement detecting disc includes a substrate and reagent blocks. The substrate includes fan-shaped blades with spoke-like arrangement and a handle, and a ring coupled with the blade by the handle. Each of the blades has a front side and a back side, and an identification code is set on the front side of each of the blades. The corresponding reagent block is pasted at the position of the corresponding identification code. The identification codes are set on the blades for a detecting device to identify types of excrement and detection items, and to choose appropriate wavelength. The center of the ring is formed with a slot. The slot is coupled with a corresponding component of the detecting device. The corresponding component of the detecting device is fixed to a motor shaft for completion of transferring and transporting a test strip, rotating, and completion scanning. The slot is matched with an mechanical arm for clamping. It is convenient for clamping suction or negative-pressure suction of the mechanical arm to the ring, and beneficial to automation. The reagent blocks include a chemical reagent block, an enzymatic reagent block, an immunological reagent block, a biochip reagent block, and a background control reagent block.

The chemical reagent block includes bilirubin, cholesterol, ketone body, specific gravity, pH value, nitrite, protein, starch, fat, and/or inorganic salt. The enzymatic reagent block includes glucose, hemoglobin, and/or white blood cells. The immunological reagent block includes hemoglobin, transferrin, pathogenic microorganism, and/or hormone. The biochip reagent block includes a microbial gene chip and/or a cancer cell gene chip.

Each of the blades has a width of 3 mm to 6 mm and a length of 10 mm to 20 mm. The blades are spaced apart from each other by not less than 2.5 mm. There is no direct contact between the blades to avoid interference from reagents of different detection items. The handle has a width of 2.5 mm and a length of 2.5 mm. The ring is disc-shaped with a diameter of 10 mm to 15 mm and can be rotated, thereby reducing the volume of the detecting device for transmission and scanning, and shortening the detection time. Each of the blades is further divided into a plurality of troughs for containing a liquid. The troughs are composed of grooves and frames on the blades. The troughs are installed with the reagent blocks or a blank therein. The blank is used for identifying, classifying, and counting of excrement samples.

The fan-shaped blades with spoke-like arrangement may be further formed into formed as three-dimensional structures having multi-layered fan-shaped blades. The layers of the blades are spaced apart from each other by 5 mm. The multi-layered blades are arranged alternately and correspondingly increasing the number of the blades and the number of the reagent blocks.

An outer edge of the ring is further provided with a socket. The socket is adapted to receive the handle. The handle is insertable and replaceable, modular-designed. The detection items are randomly combined according to needs thereby saving resources.

Each of the reagent blocks includes an item detection reagent block and a background control reagent block. Each detection item is installed with negative control, to eliminate the colors of the excrements itself, the backgrounds of reagents, the influences of dyeing of the different reagent blocks, and to improve the reliability of the detection results. In addition, a standard control of the detected component is set if necessary to achieve accurate quantitative detection. The item detection reagent blocks and the background control reagent blocks are spaced apart by 2.5 mm and arranged in series or in parallel on the blades. The background control reagent block can be a quality control reagent block. The background control reagent block is shared. Two or more item detection reagent blocks can be provided in the same blade to save spaces and to accommodate more detection items.

The item detection reagent block includes urine detection, stool detection, saliva detection, prostatic fluid detection, semen detection, milk detection, and human body drainage detection.

The reagent blocks are mounted on the blades with spoke-like arrangement and arranged in the order of pH values of the reagents. The pH values are arranged in a circular order as from 3.0, 4.0, 5.0, 6.0, 7.0, 8.0, 7.0, 6.0, 5.0 to 4.0. It is completely avoided that the mutual contacts and pollutions between the reagents and the changes of pH conditions which brings about unrealistic coloring by reactions. The width of the blades can be increased or decreased.

A method for manufacturing an excrement detecting disc includes the steps of: preparing a substrate, preparing a large-sized adsorption reagent carrier, cutting the large-sized adsorption reagent carrier into a reagent block, and bonding and packaging the reagent block to the substrate. Preparing the substrate includes three-dimensionally designing the substrate, designing a mold, PVC injection molding, and spraying an identification code. Preparing the large-sized adsorption reagent carrier includes preparing a detection reagent and a large carrier adsorption reagent. Bonding the reagent blocks to the substrate is making the reagent blocks pasted on a front side of a blade of the substrate.

By the above technical means, a detecting disc made by the method for manufacturing the excrement detecting disc of the present disclosure can accommodate more detection items since the reagent blocks are attached to the fan-shaped blades with spoke-like arrangement. At the same time, the spaces between the blades are used to avoid mutual interference between reagents of different detection items. The personalized detection items and general detection items are mashed up and coexist in the same detecting disc to improve work efficiency. Each of the detection items is set with a negative control to improve the reliability of the detection results.

### BRIEF DESCRIPTION OF THE DRAWINGS

FIG. 1 is a schematic view of a substrate;

FIG. 2 is a schematic view showing the bonding of the detection reagent blocks and the background control reagent blocks to the blades;

FIG. 3 is a schematic view showing the couplings of a slot to a detecting device;

FIG. 4A is a schematic view of the first working position of the mechanical arm clamping a detecting disc;

FIG. 4B is a schematic view of the second working position of the mechanical arm clamping a detecting disc;

FIG. 4C is a schematic view of the third working position of the mechanical arm clamping a detecting disc;

FIG. 5 is a schematic view of a socket on a ring;

FIG. 6 is a schematic view of a pluggable blade and a handle;

FIG. 7 is a schematic view showing the insertion of a blade and a handle into a socket of a ring;

FIG. 8 is a schematic view of a substrate composed of multi-layered blades;

FIG. 9 is a schematic view showing a detection reagent block and a background control reagent block arranged in series on a blade;

FIG. 10 is a schematic view showing two detection reagent blocks and the same background control reagent block arranged in series on a blade;

FIG. 11 is a schematic view showing the troughs of a detection reagent block and a background control reagent block arranged in series on a blade;

FIG. 12 is a schematic view showing the reagent blocks on the blade circularly arranged according to the pH value; and

FIG. 13 is a schematic view of a process for producing a detecting disc.

### DETAILED DESCRIPTION OF THE PREFERRED EMBODIMENTS

For a detailed description of the technical contents, structural features, objects and advantages of the present disclosure, the embodiments will be described in detail below with reference to the accompanying drawings.

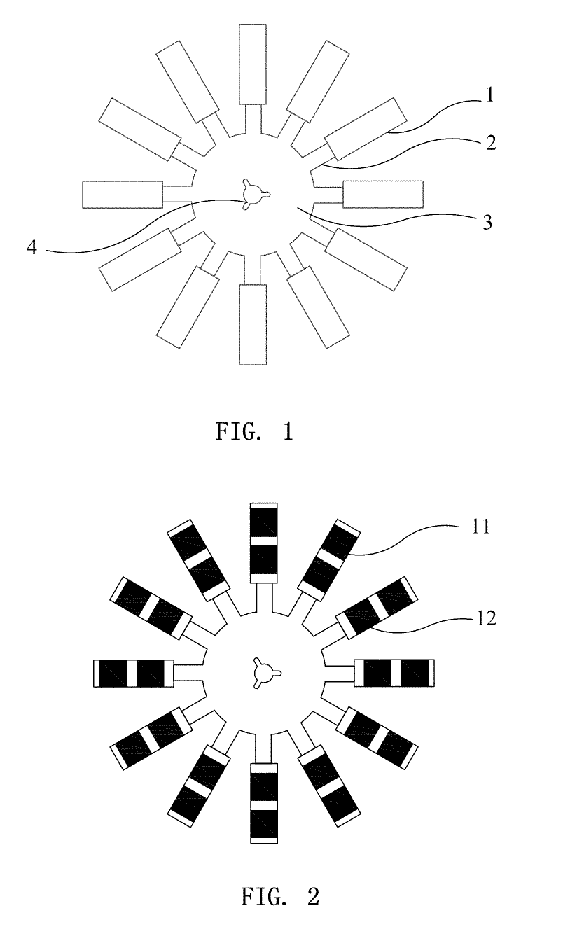

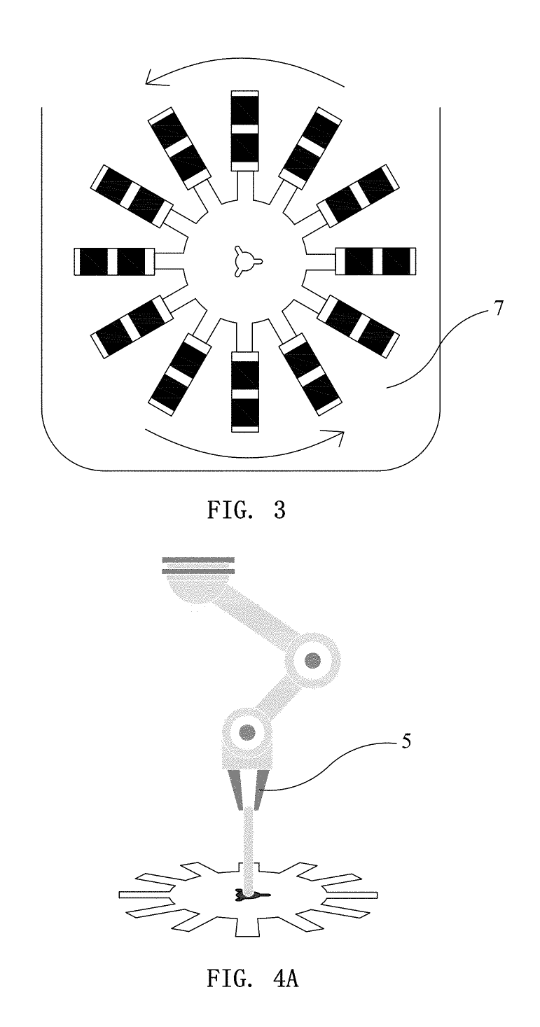

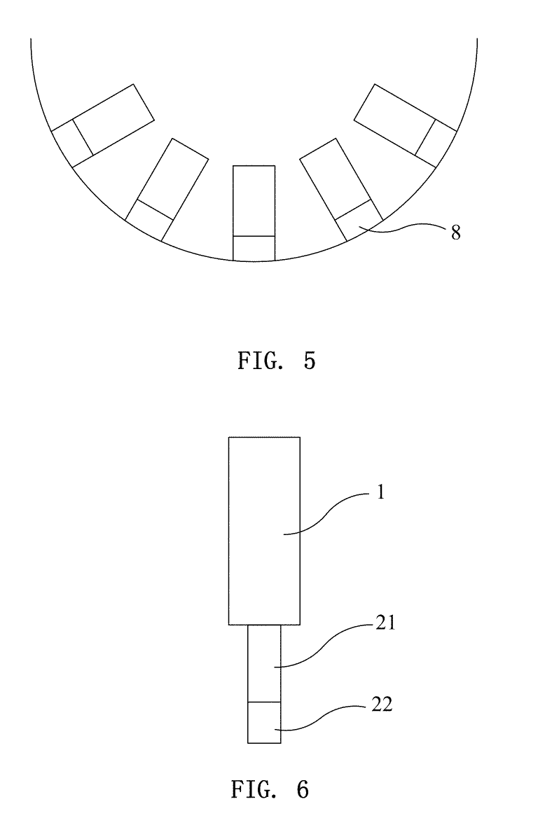

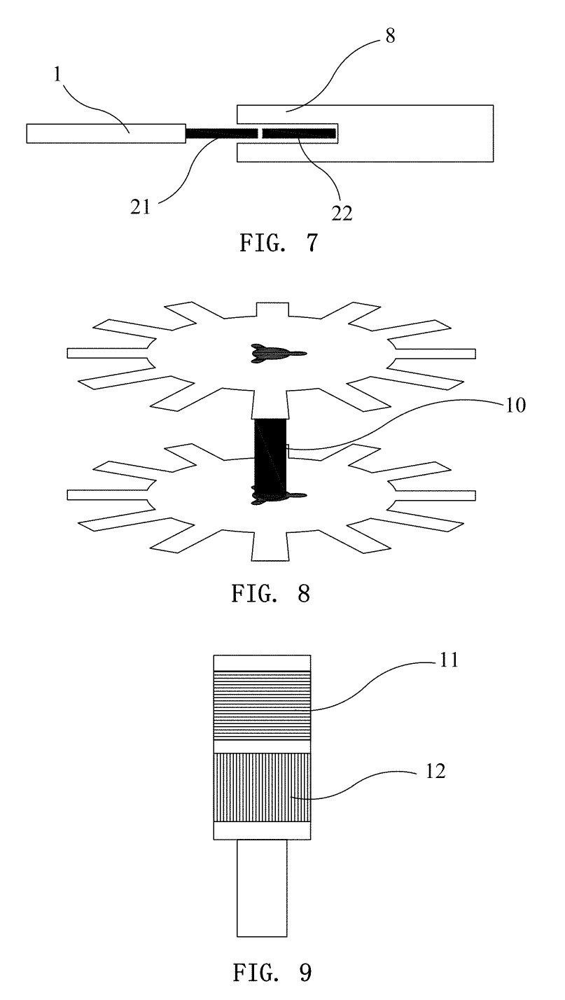

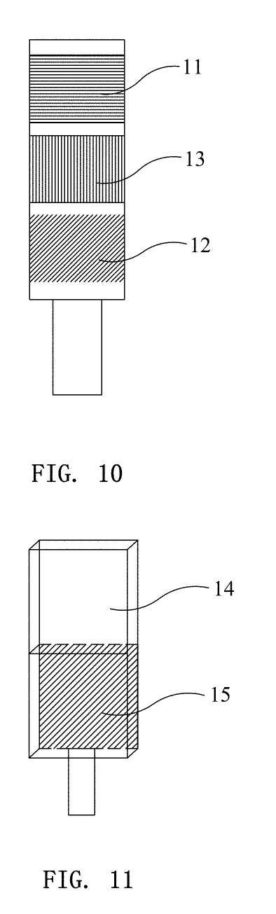

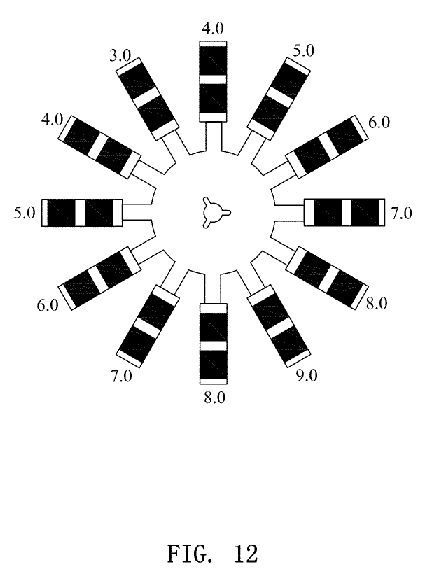

As shown in FIG. 1 to FIG. 12, the present disclosure discloses an excrement detecting disc, which includes a substrate and reagent blocks.

The substrate includes fan-shaped blades 1 with spoke-like arrangement and a handle 2, and a ring 3. The blade 1 and the ring 3 are coupled by the handle 2. Each of the blades 1 has a front side and a back side

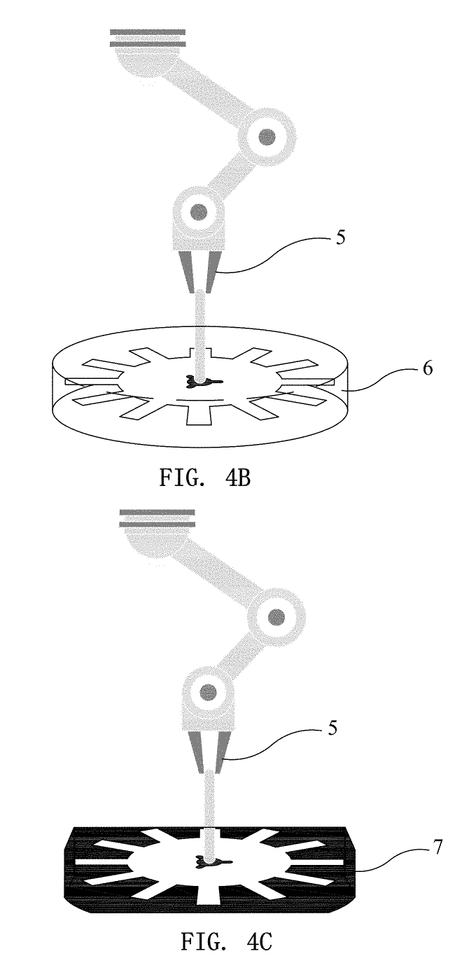

An identification code is set on the front side of each of the blades 1. The corresponding reagent block 11 is pasted at the position of the corresponding identification code. The identification codes are set on the blades 1 for a detecting device to identify types of excrements and detection items and to choose appropriate wavelength. The center of the ring 3 is formed with a slot 4. The slot 4 is coupled with a corresponding component of the detecting device 7. The corresponding component of the detecting device 7 is fixed to a motor shaft for completion of transferring and transporting the testing disc, rotating, and completion of scanning. FIG. 4A to 4C depict the transfer and transportation of the detecting disc. The rotation is carried by the rotation of the motor. During the rotation of the detecting disc, the fixed detecting device obtains the detection data by photoelectric colorimetry.

The slot 4 is matched with a mechanical arm 5 for clamping. The mechanical arm 5 is configured to suck the ring 3 or clamp the slot 4. The mechanical arm 5 can perform suction by negative pressure. Clamping the detecting disc is carried out in the first working position, soaking the detecting disc into an excrement sample container 6 is carried out in the second working position, and taking the detecting disc out of the excrement sample container 6 and sending into the detecting device 7 is carried out in the third working position, which are all performed through automate operations.

The reagent blocks include a chemical reagent block, an enzymatic reagent block, an immunological reagent block, a biochip reagent block, an item detection reagent block 11 and a background control reagent block 12. The chemical reagent block includes bilirubin, cholesterol, ketone body, specific gravity, pH value, nitrite, protein, starch, fat, and/or inorganic salt. The enzymatic reagent block includes glucose, hemoglobin, and/or white blood cells. The immunological reagent block includes hemoglobin, transferrin, pathogenic microorganism, and/or hormone. The biochip reagent block includes a microbial gene chip and/or a cancer cell gene chip.

Each of the blades 1 has a width of 3 mm to 6 mm and a length of 10 mm to 20 mm. The blades 1 are spaced apart from each other by not less than 2.5 mm. There is no direct contact between the blades 1 to avoid interference from reagents of different detection items.

The handle 2 has a width of 2.5 mm and a length of 2.5 mm. The ring 3 is disc-shaped with a diameter of 10 mm-15 mm.

The substrate has a diameter of 50 mm and can be rotated to reduce the volume of detecting device 7 for transmission and scanning and to shorten the detection time, that is, the detecting disc according to the disclosure accommodates as many detection items. Compared with the conventional long test strip, the detecting disc of the present disclosure is designed in the shape of a disc. To a certain extent, the length of the scanning of the detecting device can be reduced. Compared with the forward and backward motions for scanning of the conventional long test strip, the disc-shaped structure designed according to the present disclosure carries out scanning by rotation. To a certain extent, the detection time can be shortened.

The outer edge of the ring 3 is further provided with a socket 8. The socket 8 is adapted to receive the handle 2. The handle 2 has a length of 5 mm, and an insertable socket section 22 of the handle 2 has a length of 2.5 mm. The handle 2 is pluggable, detachable, and replaceable with the socket 8, achieving modular design. The detection items are randomly combined according to needs thereby saving resources.

The fan-shaped blades 1 with spoke-like arrangement may be further formed as three-dimensional structures having multi-layered fan-shaped blades. Each of the layers of the blades 1 is linked by a rotor 10. The rotor 10 is pluggable with the slot 4. The layers of the blades 1 are spaced apart from each other by 5 mm. The multi-layered blades 1 are arranged alternately and correspondingly increasing the number of the blades 1 and the number of the reagent blocks 11. The alternate arrangement will allow the reagent blocks provided in the blades 1 to be detected by the detecting device 7.

In this embodiment, each of the reagent blocks includes an item detection reagent block 11 and a background control reagent block 12. Each detection item is installed with negative control, to eliminate the colors of the excrements itself, the backgrounds of reagents, the influences of dyeing of the different reagent blocks, and to improve the reliability of the detection results. In addition, a standard control of the detected component is set if necessary to achieve accurate quantitative detection. The item detection reagent block 11 and the background control reagent block 12 are spaced apart by 2.5 mm and arranged in series or in parallel. The background control reagent block 12 can be shared. A third item detection reagent block 13 can be provided in the same blade 1 to save spaces and to accommodate more detection items.

The detection items include urine detection, stool detection, saliva detection, prostatic fluid detection, milk detection, and human drainage detection. The urine detection includes urine specific gravity, pH, vitamin C, nitrite, protein, albumin, leukocytes, occult blood, creatinine, urobilinogen, bilirubin, glucose, ketone bodies, hormones, inorganic salts, and other metabolites; the stool test includes feces: pH, white blood cells, occult blood (hemoglobin, transferrin), cancer cells ingredients, fecal urobilin, stercobilinogen, glucose, protein, starch, fat, pathogenic microorganisms, and intestinal flora.

The reagent blocks are mounted on the blades with spoke-like arrangement and arranged in the order of pH values of the reagents. The pH values are arranged in a circular order as from 3.0, 4.0, 5.0, 6.0, 7.0, 8.0, 7.0, 6.0, 5.0 to 4.0. It is completely avoided that the mutual contacts and pollutions between the reagents and the changes of pH conditions which brings about unrealistic coloring by reactions.

The width of the blades can be increased or decreased. The blade 1 can also be divided into a trough 14 for the detection item and a background control trough 15 to contain a liquid; the trough 14 is composed of a groove and a frame on the blade 1. A reagent block is arranged at the bottom of the trough 14 to detect the components of the excrement sample by enzymatic, chemical or immunological methods, or by a biochip method. The trough 14 can also be a blank, to carry samples, used for identifying, classifying, and counting of excrement samples.

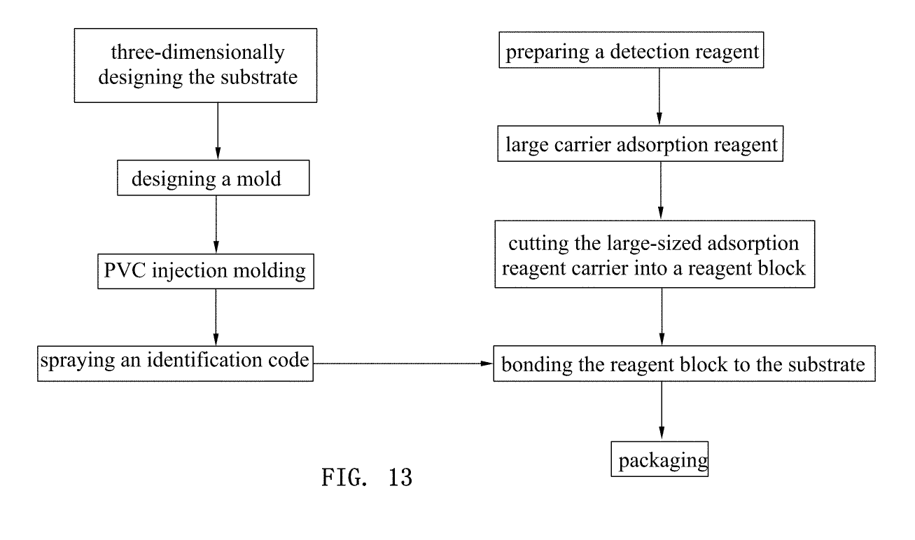

As shown in FIG. 13, a method for manufacturing an excrement detecting disc includes the steps of: preparing a substrate, preparing a large-sized adsorption reagent carrier, cutting the large-sized adsorption reagent carrier into a reagent block, bonding the reagent block to the substrate, and packaging. Preparing the substrate includes three-dimensionally designing the substrate, designing a mold, PVC injection molding, and spraying an identification code. Preparing the large-sized adsorption reagent carrier includes preparing a detection reagent and a large carrier adsorption reagent. Bonding the reagent block to the substrate is making the reagent block paste on a front side of a blade of the substrate.

The above is only the embodiment of the present disclosure is not intended to limit the technical scope of the present disclosure. Therefore, any minor alterations, equivalent changes and modifications made to the above embodiments in accordance with the technical spirit of the present invention still belong to the present invention, within the scope of the technical solution.

What is claimed is:

1. An excrement detecting disc, comprising:
a substrate comprising fan-shaped blades with spoke-like arrangement and a handle, and a ring coupled with the blade by the handle; and
and reagent blocks;
wherein each of the blades has a front side and a back side, an identification code is set on the front side of each of the blades, and a corresponding one of the reagent blocks is pasted at the position of the corresponding identification code; a center of the ring is formed with a slot, and the slot is coupled with a corresponding component of a detecting device; the corresponding component of the detecting device is fixed to a motor shaft; the reagent blocks comprise a chemical reagent block, an enzymatic reagent block, an immunological reagent block, a biochip reagent block, and a background control reagent block.

2. The excrement detecting disc according to claim 1, wherein each of the blades has a width of 3 mm to 6 mm and a length of 10 mm to 20 mm, the blades are spaced apart from each other by not less than 2 5 mm; the handle has a width of 2.5 mm and a length of 2.5 mm; and the ring is disc-shaped with a diameter of 10 mm to 15 mm.

3. The excrement detecting disc according to claim 1, wherein each of the blades is divided into a plurality of troughs; the troughs are composed of grooves and frames on the blades and are installed with the reagent blocks or a blank therein; and the blank is used for identifying, classifying, and counting of excrement samples.

4. The excrement detecting disc according to claim 1, wherein the fan-shaped blades with spoke-like arrangement is further formed into multi-layered fan-shaped blades having three-dimensional structures, the layers of the blades are spaced apart from each other by 5 mm, and the multilayer blades are arranged alternately and correspondingly increasing the number of the blades and the number of the reagent blocks.

5. The excrement detecting disc according to claim 1, wherein an outer edge of the ring is provided with a socket, the socket is adapted to receive the handle, and the handle is pluggable and replaceable.

6. The excrement detecting disc according to claim 1, wherein each of the reagent blocks comprises an item detection reagent block and a background control reagent block; the item detection reagent block and the background control reagent block are spaced apart by 2.5 mm and arranged in series or in parallel on each of the blades; the background control reagent block is a quality control reagent block; the background control reagent block is shared, and the same blade is provided with two or more item detection reagent blocks.

7. The excrement detecting disc according to claim 6, wherein the item detection reagent block comprises urine detection, stool detection, saliva detection, prostatic fluid detection, semen detection, milk detection, and human body drainage detection.

8. The excrement detecting disc according to claim 1, wherein the reagent blocks are mounted on the blades with spoke-like arrangement and arranged in the order of pH values of reagents, and the order of pH values are circularly arranged, from small to large, then small.

9. A method for manufacturing an excrement detecting disc, comprising the steps of: preparing a substrate, preparing a large-sized adsorption reagent carrier, cutting the large-sized adsorption reagent carrier into a reagent block, bonding the reagent block to the substrate, and packaging;
wherein the step of preparing the substrate comprises three-dimensionally designing the substrate, designing a mold, PVC injection molding, and spraying an identification code; the step of preparing the large-sized adsorption reagent carrier comprises preparing a detection reagent and a large carrier adsorption reagent;
wherein bonding the reagent block to the substrate is making the reagent block paste on a front side of a blade of the substrate.
